(19) United States
(12) Patent Application Publication
(10) Pub. No.: US 2020/0289088 A1
(43) Pub. Date: Sep. 17, 2020

(54) APPARATUS FOR AND METHOD OF IMPROVING ROTATION STABILITY AND EXPANDABILITY OF INTEGRATED SCANNING SYSTEM

(71) Applicant: POSTECH ACADEMY-INDUSTRY FOUNDATION, Pohang-si (KR)

(72) Inventors: Jae Woo KIM, Pohang-si (KR); Chul Hong KIM, Pohang-si (KR); Yong Min KIM, Pohang-si (KR); Seong Hee CHO, Pohang-si (KR); Joong Ho AHN, Pohang-si (KR)

(21) Appl. No.: 16/065,699

(22) PCT Filed: Apr. 17, 2018
(86) PCT No.: PCT/KR2018/004450

§ 371 Date: Jun. 22, 2018

(30) Foreign Application Priority Data
Dec. 14, 2017 (KR) 10-2017-0172551

(51) Int. Cl. A61B 8/00; A61B 5/00; G02B 6/36; F16H 3/00; H01R 39/08

(52) U.S. Cl. CPC A61B 8/445; A61B 5/0086; H01R 39/08; G02B 6/3604; F16H 3/006; A61B 5/0095

(57) ABSTRACT

According to an apparatus for and a method of improving rotation stability and expandability of an integrated scanning system provided in the present invention, an intravascular photoacoustic scanning system is provided with a slip ring and an optical rotary joint disposed independently on the same shaft, and an intermediate shaft disposed with the slip ring and the optical rotary joint in parallel to transfer torque of a motor, whereby it is possible to expand a configuration of the system with an additional component, and to obtain stable rotation by independent drive control for power transfer of components such as the slip ring and the optical rotary joint.

### TECHNICAL FIELD

The present invention relates to an apparatus for and a method of improving rotation stability and expandability of an integrated scanning system. More specifically, the present invention relates to an apparatus for and a method of improving rotation stability and expandability of an integrated scanning system in which an intravascular photoacoustic scanning system is configured with an intermediate shaft inserted therein to expand a configuration of the system and to obtain stable rotation by independent drive control for components.

### BACKGROUND ART

In general, cardiovascular-related diseases account for more than one-third of causes of deaths worldwide and have attracted much attention from academia and the medical community. There are medical imaging methodologies for vascular image catheters such as intravascular ultrasound, intravascular near-infrared spectroscopy, and intravascular optical coherence tomography, which are used for the diagnosis of vascular diseases, and these methodologies has been developed for the diagnosis and treatment of the diseases and have been utilized in clinical practice.

Intravascular ultrasound is a technique for acquiring a tomographic image of a blood vessel by inserting a catheter-type device into a blood vessel, and is very useful for an intravascular or intracardiac procedure. Thus, intravascular ultrasound is still the most widely used intravascular image methodology in hospitals. Ultrasonography can show cross-sectional images of tissue cells in real time using ultrasound, which can quantitatively and qualitatively distinguish the types, lengths, and states of lesions in three dimensions. In addition, intravascular near-infrared imaging technology is a commercialized technology that uses near-infrared light to detect a presence of lipids in an inner wall of a blood vessel by spectroscopic method and it has been developed as a single catheter combined with intravascular ultrasound recently.

Optical imaging methods based on fiber optic technology used in the field of medicine include optical coherence tomography (OCT), angioscopy, near-infrared spectroscopy, Raman spectroscopy, and fluorescence spectroscopy. In OCT, a catheter-type device is used as in the case of an intravascular ultrasound, and is inserted into a blood vessel to transmit light and analyzes the reflected light to acquire a tomographic image of the blood vessel.

As described above, medical technologies such as intravascular ultrasound, intravascular near-infrared spectroscopy, and intravascular optical coherence tomography for diagnosis and treatment of cardiovascular-related diseases are used for diagnosis and treatment of diseases. However, only limited information can be obtained with a single technology, which makes accurate diagnosis difficult. Accordingly, various types of intravascular catheters have been combined to accurately diagnose lesions in recent years. A scanning system of a medical device for the diagnosis and treatment of a blood vessel includes a component requiring torque of a motor for a specific purpose such as a slip ring and an optical rotary joint, and an additional component is directly fastened the component receiving the torque of the motor such that the component receives power directly. Accordingly, when rotation axes of a rotating component that receives torque directly and dependent components receiving the torque from the rotating component are not aligned, a center of mass of the dependent components are displaced from the rotation axis, which causes very severe vibration during rotation and thus the entire scanning system vibrates, whereby quality of the image becomes deteriorated. In addition, in the related art, an adapter suitable for a structure is required to be manufactured between the components, and since an order of the components is fixed when the adapter is manufactured, it is difficult to add new components. Further, in a scanning system of a medical device in the related art, when a speed of the motor is not constant, the speed change is directly transferred to a drive, causing that an image is rotated clockwise or counterclockwise. Korean Patent No. 10-1397272, Korean Patent No. 10-1737440, and Korean Patent Application Publication No. 10-2014-0133372 are examples of related art documents.

### DISCLOSURE

### Technical Problem

Accordingly, the present invention has been made keeping in mind the above problems occurring in the related art, and an object of the present invention is to provide an apparatus for and a method of improving rotation stability and expandability of an integrated scanning system. The system, which is an intravascular photoacoustic scanning system, is provided with a slip ring and an optical rotary joint disposed independently on the same shaft and provided with an intermediate shaft disposed with the slip ring and the optical rotary joint in parallel to transfer a rotational force, that is, torque, whereby it is possible to expand a configuration of the system with an additional component, and to obtain stable rotation by independent drive control for power transfer of components such as the slip ring and the optical rotary joint.

In addition, another object of the present invention is to provide an apparatus for and a method of improving rotation stability and expandability of an integrated scanning system in which a slip ring and an optical rotary joint receive torque individually by an intermediate shaft disposed in parallel in the scanning system such that there is no need to directly fasten each component, and even if rotational axes of the components are misaligned from each other, stable rotation can be achieved only if rotational speeds thereof are same, and in particular, the conventional vibration problem that occurred when center axes are misaligned due to mechanical errors can be fundamentally solved.

Furthermore, still another object of the present invention is to provide an apparatus for and a method of improving rotation stability and expandability of an integrated scanning system in which any one of pulleys of a motor and of a main drive part disposed on an intermediate shaft further includes an unidirectional bearing such that even if torque of the motor is changed continually, power is transferred in only one direction by the unidirectional bearing, thereby minimizing quality deterioration of an image due to nonuniformity of a motor speed.

### Technical Solution

In order to accomplish the above object, the present invention provides

an apparatus for improving rotation stability and expandability of an integrated scanning system, the apparatus including:

a slip ring configured to prevent entanglement of an electric wire;

an optical rotary joint configured to prevent entanglement of an optical fiber;

a motor configured to transfer torque to the slip ring and the optical rotary joint; and

an intermediate shaft configured to transfer the torque of the motor to the slip ring and the optical rotary joint, and to synchronize rotations of the motor, the slip ring, and the optical rotary joint.

The slip ring may include

the electric wire, which is used to transmit and receive an electric signal from an ultrasonic pulser/receiver, and

the electric wire

may be connected to an ultrasonic transducer of the integrated scanning system and serves to transmit a photoacoustic signal obtained from the ultrasonic transducer to an image processing unit.

The optical rotary joint may include

a multi-mode optical fiber, which is used to transmit a light signal, and

the optical fiber

serves to transmit a laser toward an ultrasonic transducer of the integrated scanning system.

The intermediate shaft

may be disposed with the slip ring and the optical rotary joint in parallel.

The intermediate shaft may be configured with

a main drive part disposed on the intermediate shaft to receive torque from a motor pulley of the motor; and

component drive parts disposed on the intermediate shaft on which the main drive part is disposed to transfer the torque transferred from the main drive part to the slip ring and the optical rotary joint respectively.

The slip ring and the optical rotary joint

may be configured to be disposed independently to receive the torque from the respective component drive parts disposed on the intermediate shaft.

The slip ring and the optical rotary joint

may be configured to be disposed independently to prevent vibration and thus improve stability of rotation such that each center of mass of the slip ring and the optical rotary joint exists on a rotation axis thereof.

The intermediate shaft

may be configured to allow expansion of the integrated scanning system by adding a new component drive part to transfer the torque to an additional component.

The intermediate shaft

may serve to improve uniformity of a motor speed by adjusting a gear ratio between the motor pulley of the motor and a pulley of the main drive part.

Any one of the motor pulley of the motor and the pulley of the main drive part may further include

an unidirectional bearing that transfers power during acceleration of the motor, but serves as a bearing during deceleration of the motor so as not to transfer power, thereby improving the uniformity of the motor speed.

In order to accomplish the above object, the present invention provides

an apparatus for improving rotation stability and expandability of an integrated scanning system, the apparatus including:

a slip ring configured to prevent entanglement of an electric wire;

an optical rotary joint disposed on a same shaft with the slip ring and configured to prevent entanglement of an optical fiber;

a motor provided with a motor pulley and configured to transfer torque to the slip ring and the optical rotary joint; and

an intermediate shaft configured to transfer the torque of the motor to the slip ring and the optical rotary joint, and disposed with the slip ring and the optical rotary joint in parallel to synchronize rotations of the motor, the slip ring, and the optical rotary joint,

wherein the intermediate shaft includes

a main drive part disposed on the intermediate shaft to receive the torque from the motor pulley of the motor, and component drive parts disposed on the intermediate shaft on which the main drive part is disposed to transfer the torque transferred from the main drive part to the slip ring and the optical rotary joint respectively, and

timing belts connect the motor pulley and a pulley of the main drive part to each other, and connect pulleys of the component drive parts and pulleys of the slip ring and the optical rotary joint to each other to transfer the torque.

The intermediate shaft

may be configured to allow expansion of the integrated scanning system by adding a new component drive part to transfer the torque to an additional component.

Any one of the motor pulley of the motor and the pulley of the main drive part may further include

an unidirectional bearing that transfers power during acceleration of the motor, but serves as a bearing during deceleration of the motor so as not to transfer power, thereby improving the uniformity of the motor speed.

In order to accomplish the above object, the present invention provides

a method of improving rotation stability and expandability of an integrated scanning system including a slip ring, an optical rotary joint, a motor, and an intermediate shaft, the method including:

(1) a step in which a pulley of a main drive part (141) of the intermediate shaft receives torque through a motor pulley of the motor;

(2) a step in which a plurality of component drive parts disposed and fixed on the intermediate shaft rotates on the intermediate shaft by the torque of the main drive part; and

(3) a step in which the plurality of component drive parts disposed and fixed on the intermediate shaft transfers the torque to the slip ring and the optical rotary joint respectively.

The intermediate shaft

may be disposed with the slip ring and the optical rotary joint in parallel.

The intermediate shaft

may synchronize rotations of the motor, the slip ring, and the optical rotary joint.

The slip ring and the optical rotary joint

may be configured to be disposed independently on the same shaft to receive torque from the respective component drive parts disposed on the intermediate shaft.

The intermediate shaft

may be configured to allow expansion of the integrated scanning system by adding a new component drive part to transfer the torque to an additional component.

The intermediate shaft

may serve to improve uniformity of a motor speed by adjusting a gear ratio between the motor pulley of the motor and a pulley of the main drive part.

Any one of the motor pulley of the motor and the pulley of the main drive part may further include

an unidirectional bearing that transfers power during acceleration of the motor, but serves as a bearing during deceleration of the motor so as not to transfer power, thereby improving the uniformity of the motor speed.

### Advantageous Effects

According to the apparatus for and the method of improving rotation stability and expandability of the integrated scanning system of the present invention, the intravascular photoacoustic scanning system is provided with the slip ring and the optical rotary joint disposed independently on the same shaft, and the intermediate shaft disposed with the slip ring and the optical rotary joint in parallel to transfer torque of the motor, whereby it is possible to expand the configuration of the system with an additional component, and to obtain stable rotation by independent drive control for power transfer of components such as the slip ring and the optical rotary joint.

In addition, according to the present invention, the slip ring and the optical rotary joint receives torque individually by the intermediate shaft disposed in parallel in the scanning system such that there is no need to directly fasten each component, and even if the rotational axes of the components are misaligned from each other, stable rotation can be achieved only if rotational speeds thereof are same, and in particular, the conventional vibration problem that occurred when the center axes are misaligned due to mechanical errors can be fundamentally solved.

Furthermore, in the present invention, any one of the pulleys of the motor and of the main drive part disposed on the intermediate shaft further includes the unidirectional bearing such that even if torque of the motor is changed continually, power is transferred in only one direction by the unidirectional bearing, thereby minimizing quality deterioration of an image due to nonuniformity of the motor speed.

### DESCRIPTION OF DRAWINGS

FIG. 1 is a diagram showing a configuration of an apparatus for improving rotation stability and expandability of the integrated scanning system according to an embodiment of the present invention;

FIGS. 2A and 2B depict diagrams comparatively showing configurations of the apparatus for improving rotation stability and expandability of the integrated scanning system according to the embodiment of the present invention before and after using an intermediate shaft, respectively;

FIG. 3 is a diagram showing an example when a gear ratio of the apparatus for improving rotation stability and expandability of the integrated scanning system according to the embodiment is equal;

FIG. 4 is a diagram showing an example when gear ratio of the apparatus for improving rotation stability and expandability of the integrated scanning system according to the embodiment is high;

FIG. 5 is a diagram showing a configuration of the apparatus for improving rotation stability and expandability of the integrated scanning system according to the embodiment before using an unidirectional bearing;

FIG. 6 is a diagram showing a configuration of the apparatus for improving rotation stability and expandability of the integrated scanning system according to the embodiment after using an unidirectional bearing; and

FIG. 7 is a flow chart showing a driving sequence of a method of improving rotation stability and expandability of the integrated scanning system according to an embodiment of the present invention.

### DESCRIPTION OF REFERENCE NUMERALS IN THE DRAWINGS

100: apparatus for improving rotation stability and expandability according to an embodiment of the present invention
110: slip ring
111: electric wire
120: optical rotary joint
121: optical fiber
130: motor
131: motor pulley
140: intermediate shaft
141: main drive part
142: component drive part
143: unidirectional bearing
S110: step in which a pulley of a main drive part of an intermediate shaft receives torque through a motor pulley of a motor
S120: step in which a plurality of component drive parts disposed and fixed on the intermediate shaft rotates on the intermediate shaft by the torque of the main drive part
S130: step in which the plurality of component drive parts disposed and fixed on the intermediate shaft transfers the torque to the slip ring and the optical rotary joint

### BEST MODE

Hereinbelow, preferred embodiments of the present invention will be described in detail with reference to the accompanying drawings such that the invention can be easily embodied by one of ordinary skill in the art to which this invention belongs. It is to be noted that, when the functions of conventional elements and the detailed description of elements related with the present invention may make the gist of the present invention unclear, a detailed description of those elements will be omitted. In addition, the same reference numerals will be used throughout the drawings and the description to refer to the same or like elements or parts.

Furthermore, it will be understood that when an element is referred to as being “coupled” or “connected” to another element, it can be directly coupled or connected to the other element or intervening elements may be present therebetween. It will be further understood that the terms “comprise”, “include”, “have”, etc. when used in this specification, specify the presence of stated features, integers, steps, operations, elements, components, and/or combinations of them but do not preclude the presence or addition of one or more other features, integers, steps, operations, elements, components, and/or combinations thereof.

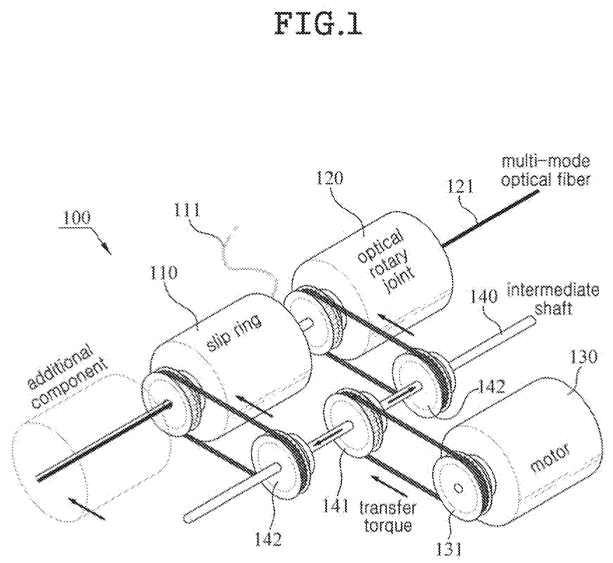

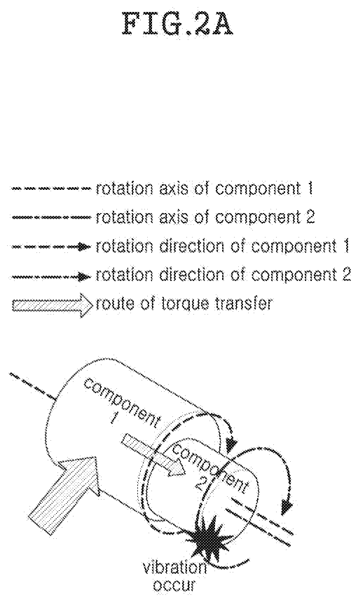

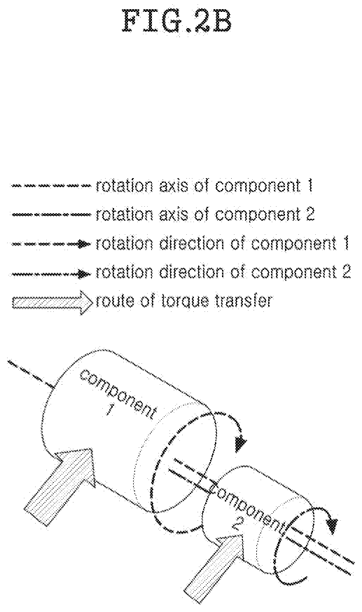

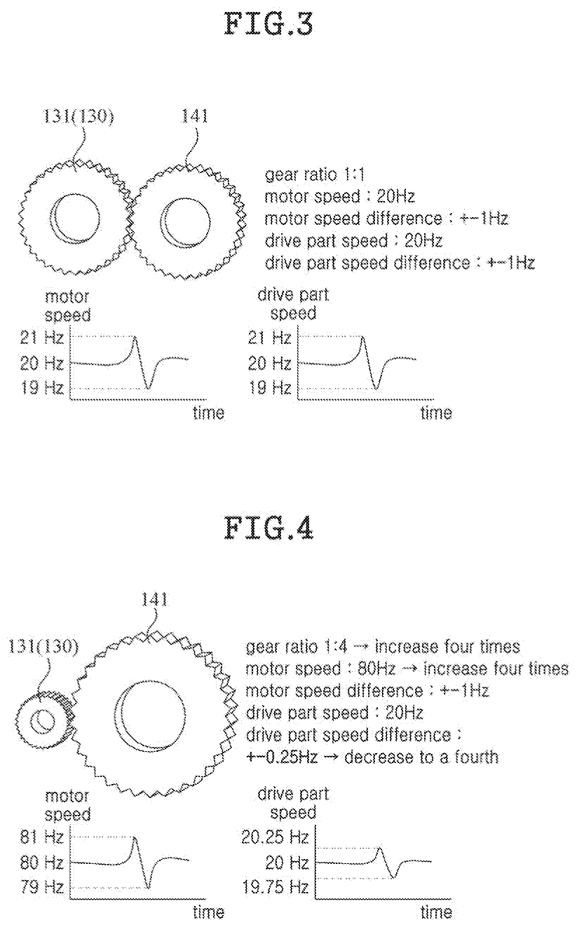

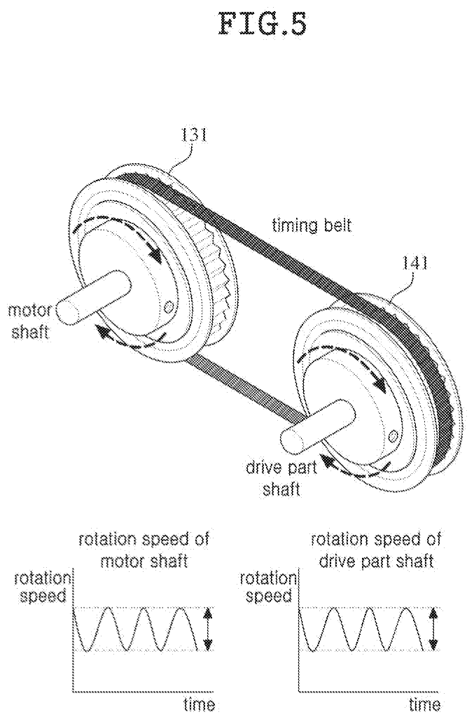

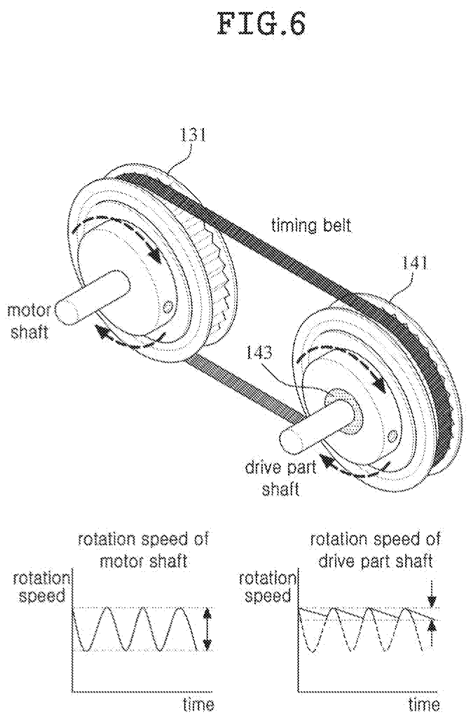

FIG. 1 is a diagram showing a configuration of apparatus for improving rotation stability and expandability of the integrated scanning system according to an embodiment of the present invention; FIGS. 2A and 2B depict diagrams comparatively showing configurations of the apparatus for improving rotation stability and expandability of the integrated scanning system according to the embodiment of the present invention before and after using an intermediate shaft, respectively; FIG. 3 is a diagram showing an example when gear ratio of the apparatus for improving rotation stability and expandability of the integrated scanning system according to the embodiment is equal; FIG. 4 is a diagram showing an example when gear ratio of the apparatus for improving rotation stability and expandability of the integrated scanning system according to the embodiment is high; FIG. 5 is a diagram showing a configuration of the apparatus for improving rotation stability and expandability of the integrated scanning system according to the embodiment before using an unidirectional bearing; and FIG. 6 is a diagram showing a configuration of the apparatus for improving rotation stability and expandability of the integrated scanning system according to the embodiment after using an unidirectional bearing. As shown in FIGS. 1 to 6, an apparatus 100 for improving rotation stability and expandability of the integrated scanning system according to an embodiment of the present invention includes a slip ring 110, an optical rotary joint 120, a motor 130, and an intermediate shaft 140.

The apparatus 100 for improving the rotation stability and expandability of the integrated scanning system according to the embodiment of the present invention is configured for use in medical devices such as intravascular ultrasound, intravascular near-infrared spectroscopy, and intravascular optical coherence tomography that images a signal obtained from an inner wall of a blood vessel while continuously rotating an inserted catheter in the blood vessel. In order to diagnose lesions with higher accuracy, a combination of medical devices has recently been developed. In this case, an electrical signal and a light signal is required to be transmitted to an end of the catheter at the same time, and continuous rotation of the catheter is essential for high-speed imaging. Accordingly, the present invention can be understood as a configuration applied to a scanning system that transmits an electrical signal and a light signal between a fixed system of a medical device and a rotating catheter. Hereinbelow, a configuration of an image processing unit of the medical device is the conventional configuration, and thus a detailed description thereof will be omitted, and the apparatus 100 for improving the rotation stability and expandability of the integrated scanning system will be described.

The slip ring 110 is configured to prevent entanglement of an electric wire 111. The slip ring 110 includes the electric wire 111, which is used to transmit and receive an electric signal from an ultrasonic pulser/receiver. The electric wire 111 is connected to an ultrasonic transducer of the integrated scanning system and serves to transmit a photoacoustic signal obtained from the ultrasonic transducer to the image processing unit. Here, the slip ring 110 is configured to be disposed independently with respect to the optical rotary joint 120, which will be described later, and to receive rotational force, that is, torque, from a corresponding component drive part 142 disposed on the intermediate shaft 140. In order to prevent vibration and thus improve stability of the rotation, the slip ring 110 is configured to be disposed independently with respect to the optical rotary joint 120 as shown in FIG. 2B such that a center of mass of the slip ring 110 exists on the rotation axis thereof.

The optical rotary joint 120 is configured to prevent entanglement of an optical fiber 121. The optical rotary joint 120 includes the multi-mode optical fiber 121, which is used to transmit a light signal. The optical fiber 121 serves to transmit a laser toward the ultrasonic transducer of the integrated scanning system. Here, the optical rotary joint 120 is configured to be disposed independently with respect to the slip ring 110 as described above, and to receive the torque from respective component drive parts 142 disposed on the intermediate shaft 140. In order to prevent vibration and thus improve stability of the rotation, the optical rotary joint 120 is configured to be disposed independently with respect to the slip ring 110 as shown in FIG. 2B such that a center of mass of the optical rotary joint 120 exists on the rotation shaft thereof.

The motor 130 is configured to transfer the torque to the slip ring 110 and the optical rotary joint 120. A configuration of the motor 130 is well known, which is a conventional power source applied to a scanning system that transmits an electric signal and a light signal between a fixed system of a medical device and a rotating catheter, and thus a detailed description thereof will be omitted.

The intermediate shaft 140 is configured to transfer the torque of the motor 130 to the slip ring 110 and the optical rotary joint 120, and to synchronize rotations of the motor 130, the slip ring 110, and the optical rotary joint 120. As shown in FIG. 1, the intermediate shaft 140 may be disposed with the slip ring 110 and the optical rotary joint 120 in parallel. Here, the intermediate shaft 140 may be configured with a main drive part 141 disposed on the intermediate shaft 140 to receive torque from a motor pulley 131 of the motor 130, and component drive parts 142 disposed on the intermediate shaft 140 on which the main drive part 141 is disposed to transfer the torque transferred from the main drive part 141 to the slip ring 110 and the optical rotary joint 120 respectively.

In addition, the intermediate shaft 140 may be configured to allow expansion of the integrated scanning system by adding a new component drive part 142 to transfer the torque to an additional component. As shown in FIG. 4, the intermediate shaft 140 may serve to improve uniformity of a motor speed by adjusting a gear ratio between the motor pulley 131 of the motor 130 and a pulley of the main drive part 141. In addition, as shown in FIG. 6, any one of the motor pulley 131 of the motor 130 and the pulley of the main drive part 141 may further include an unidirectional bearing 143 that transfers power during acceleration of the motor 130, but serves as a bearing during deceleration of the motor 130 so as not to transfer power, thereby improving the uniformity of the motor speed.

The apparatus 100 for improving the rotation stability and expandability of the integrated scanning system according to the embodiment of the present invention includes the slip ring 110 configured to prevent the entanglement of the electric wire 111, the optical rotary joint 120 disposed on the same shaft with the slip ring 110 but provided as a discrete component and configured to prevent the entanglement of the optical fiber 121, the motor 130 provided with the motor pulley 131 and configured to transfer the torque to the slip ring 110 and the optical rotary joint 120, and the intermediate shaft 140 configured to transfer the torque of the motor 130 to the slip ring 110 and the optical rotary joint 120 and disposed with the slip ring 110 and the optical rotary joint 120 in parallel to synchronize rotations of the motor 130, the slip ring 110, and the optical rotary joint 120. Here, the intermediate shaft 140 may include the main drive part 141 disposed on the intermediate shaft 140 to receive the torque from the motor pulley 131 of the motor 130, and the component drive parts 142 disposed on the intermediate shaft 140 on which the main drive part 141 is disposed to transfer the torque transferred from the main drive part 141 to the slip ring 110 and the optical rotary joint 120 respectively. In addition, timing belts connect the motor pulley 131 and the pulley of the main drive part 141 to each other, and connect pulleys of the component drive parts 142 and pulleys of the slip ring 110 and the optical rotary joint 120 to each other to transfer the torque.

Furthermore, the intermediate shaft 140 may be configured to allow expansion of the integrated scanning system by adding a new component drive part 142 to transfer the torque to an additional component. Any one of the motor pulley 131 of the motor 130 and the pulley of the main drive part 141 may further include the unidirectional bearing 143 that transfers power during acceleration of the motor 130, but serves as a bearing during deceleration of the motor 130 so as not to transfer power, thereby improving the uniformity of the motor speed.

FIGS. 2A and 2B depict diagrams comparatively showing configurations of the apparatus for improving rotation stability and expandability of the integrated scanning system according to the embodiment of the present invention before and after using an intermediate shaft, respectively. FIG. 2A is a conventional case in which two components provided in the scanning system are directly coupled, and when a center axis of a component 2 is misaligned from a rotation axis of a component 1, the center axis of the component 2 rotates around the rotation axis of the component 1 and generates a vibration. FIG. 2B is a case of the present invention in which the two components of the scanning system independently receive torque from the intermediate shaft 140. In this case, regardless of whether the rotational axis and the central axis of the two components coincide with each other, each rotation axis of the components can be maintained constantly whereby rotations can be stabilized and the quality of the image can be improved.

FIG. 3 is a diagram showing an example when gear ratio of the apparatus for improving rotation stability and expandability of the integrated scanning system according to the embodiment is equal, and FIG. 4 is a diagram showing an example when gear ratio of the apparatus for improving rotation stability and expandability of the integrated scanning system according to the embodiment is high. FIGS. 3 and 4 are diagrams comparatively showing corrections of the rotational error of an image with an increased gear ratio. In general, it is impossible to maintain a constant speed of the motor. Accordingly, an error between a motor speed and an image collection trigger occurs such that an image appears to rotate in any direction or shakes. In order to compensate, as shown in FIG. 4, when the gear ratio and the motor speed are increased in the same proportion, it is possible to reduce the vibration of the image due to the nonuniformity of the motor speed at the same rate while keeping the rotation speed of the gear of the drive constant.

FIG. 5 is a diagram showing a configuration of the apparatus for improving rotation stability and expandability of the integrated scanning system according to the embodiment without the unidirectional bearing; and FIG. 6 is a diagram showing a configuration of the apparatus for improving rotation stability and expandability of the integrated scanning system according to the embodiment with the unidirectional bearing. FIGS. 5 and 6 are diagrams showing comparative examples in which a speed difference of the drive is reduced by using the unidirectional bearing 143. The unidirectional bearing 143 is the same as a normal bearing with respect to one rotation direction, but a roller does not rotate in an opposite direction and thus transfers power. That is, when a general timing pulley is used, a speed change of the motor shaft is directly transferred to the pulley of the motor, which is finally transferred to the drive shaft through the drive pulley. As shown in FIG. 6, when the unidirectional bearing is inserted to the drive timing pulley, in a section where the motor 130 is accelerated, the roller in the bearing do not rotate, thereby transferring the power like a normal timing pulley. However, in a section where the motor 130 is decelerated, the power is applied in an opposite rotation direction and thus as the roller of the unidirectional bearing 143 begins to rotate, the power transfer is not effected and the speed of the drive shaft can be maintained.

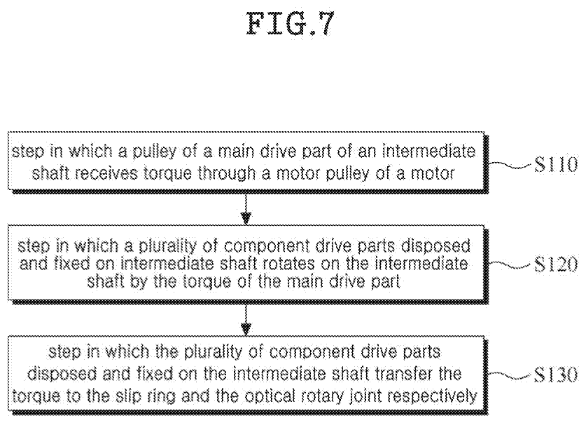

FIG. 7 is a flow chart showing a driving sequence of a method of improving rotation stability and expandability of the integrated scanning system according to an embodiment of the present invention. As shown in FIG. 7, the method of improving rotation stability and expandability of the integrated scanning system according to the embodiment of the present invention includes that the pulley of the main drive part of the intermediate shaft receives torque through the motor pulley at step S110, a plurality of component drive parts disposed and fixed on the intermediate shaft rotates on the intermediate shaft by the torque of the main drive part at step S120, and the plurality of component drive parts disposed and fixed on the intermediate shaft transfers the torque to the slip ring and the optical rotary joint respectively at step S130.

At step S110, the pulley of the main drive part 141 of the intermediate shaft 140 receives the torque through the motor pulley 131 of the motor 130. At step S120, the plurality of component drive parts 142 disposed and fixed on the intermediate shaft 140 rotates on the intermediate shaft 140 by the torque of the main drive part 141. At step S130, the plurality of component drive parts 142 disposed and fixed on the intermediate shaft 140 transfers the torque to the slip ring 110 and the optical rotary joint 120 respectively. Here, the intermediate shaft 140 is disposed with the slip ring 110 and the optical rotary joint 120 in parallel, and synchronizes rotations of the motor 130, the slip ring 110, and the optical rotary joint 120.

In addition, the slip ring 110 and the optical rotary joint 120 may be configured to be disposed independently on the same axis to receive the torque from the respective component drive part 142 disposed on the intermediate shaft 140. In addition, the intermediate shaft 140 may be configured to allow expansion of the integrated scanning system by adding a new component drive part 142 to transfer the torque to an additional component. In addition, the intermediate shaft 140 may serve to improve the uniformity of the motor speed by adjusting the gear ratio between the motor pulley 131 of the motor 130 and the pulley of the main drive part 141. Furthermore, any one of the motor pulley 131 of the motor 130 and the pulley of the main drive part 141 may further include the unidirectional bearing 143 that transfers power during acceleration of the motor 130, but serves as a bearing during deceleration of the motor 130 so as not to transfer power, thereby improving the uniformity of the motor speed.

As described above, in the apparatus for and the method of improving rotation stability and expandability of the integrated scanning system according to the embodiment of the present invention, an intravascular photoacoustic scanning system is provided with the slip ring and the optical rotary joint disposed independently on the same shaft, and provided with the intermediate shaft disposed with the slip ring and the optical rotary joint in parallel to transfer torque of the motor, whereby it is possible to expand a configuration of the system with an additional component and to obtain stable rotation by independent drive control for power transfer of components such as the slip ring and the optical rotary joint. In particular, the slip ring and the optical rotary joint receives torque individually by the intermediate shaft disposed in parallel in the scanning system such that there is no need to directly fasten each component and even if rotational axes of the components are misaligned from each other, stable rotation can be achieved only if rotational speeds thereof are same. Accordingly, the conventional vibration problem that occurred when center axes are misaligned due to mechanical errors can be fundamentally solved. Furthermore, any one of pulleys of a motor and of a main drive part disposed on the intermediate shaft further includes an unidirectional bearing such that even if torque of the motor is changed continually, power is transferred in only one direction by the unidirectional bearing, thereby minimizing quality deterioration of an image due to nonuniformity of a motor speed.

Accordingly, it should be understood that the present invention includes various modifications, additions and substitutions without departing from the scope and spirit of the invention as disclosed in the accompanying claims. The patent right of the present invention should be defined by the scope and spirit of the invention as disclosed in the accompanying claims.

1. An apparatus (100) for improving rotation stability and expandability of an integrated scanning system, the apparatus (100) comprising:
a slip ring (110) configured to prevent entanglement of an electric wire (111);
an optical rotary joint (120) configured to prevent entanglement of an optical fiber (121);
a motor (130) configured to transfer torque to the slip ring (110) and the optical rotary joint (120); and
an intermediate shaft (140) configured to transfer the torque of the motor (130) to the slip ring (110) and the optical rotary joint (120), and to synchronize rotations of the motor (130), the slip ring (110), and the optical rotary joint (120).

2. The apparatus of claim 1, wherein the slip ring (110) includes the electric wire (111), which is used to transmit and receive an electric signal from an ultrasonic pulser/receiver, and
the electric wire (111) is connected to an ultrasonic transducer of the integrated scanning system and serves to transmit a photoacoustic signal obtained from the ultrasonic transducer to an image processing unit.

3. The apparatus of claim 1, wherein the optical rotary joint (120) includes a multi-mode optical fiber (121), which is used to transmit a light signal, and
the optical fiber (121) serves to transmit a laser toward an ultrasonic transducer of the integrated scanning system.

4. The apparatus of claim 1, wherein the intermediate shaft (140) is disposed with the slip ring (110) and the optical rotary joint (120) in parallel.

5. The apparatus of claim 4, wherein the intermediate shaft (140) is configured with a main drive part (141) disposed on the intermediate shaft (140) to receive torque from a motor pulley (131) of the motor (130); and
component drive parts (142) disposed on the intermediate shaft (140) on which the main drive part (141) is disposed to transfer the torque transferred from the main drive part (141) to the slip ring (110) and the optical rotary joint (120) respectively.

6. The apparatus of claim 5, wherein the slip ring (110) and the optical rotary joint (120) are configured to be disposed independently to receive the torque from the respective component drive parts (142) disposed on the intermediate shaft (140).

7. The apparatus of claim 6, wherein the slip ring (110) and the optical rotary joint (120) are configured to be disposed independently to prevent vibration and thus improve stability of rotation such that each center of mass of the slip ring (110) and the optical rotary joint (120) exists on a rotation axis thereof.

8. The apparatus of claim 5, wherein the intermediate shaft (140) is configured to allow expansion of the integrated scanning system by adding a new component drive part (142) to transfer the torque to an additional component.

9. The apparatus of claim 8, wherein the intermediate shaft (140) serves to improve uniformity of a motor speed by adjusting a gear ratio between the motor pulley (131) of the motor (130) and a pulley of the main drive part (141).

10. The apparatus of claim 9, wherein any one of the motor pulley (131) of the motor (130) and the pulley of the main drive part (141) further includes an unidirectional bearing (143) that transfers power during acceleration of the motor (130), but serves as a bearing during deceleration of the motor (130) so as not to transfer power, thereby improving the uniformity of the motor speed.

11. An apparatus (100) for improving rotation stability and expandability of an integrated scanning system, the apparatus (100) comprising:
a slip ring (110) configured to prevent entanglement of an electric wire (111);
an optical rotary joint (120) disposed on a same shaft with the slip ring (110) and configured to prevent entanglement of an optical fiber (121);
a motor (130) provided with a motor pulley (131) and configured to transfer torque to the slip ring (110) and the optical rotary joint (120); and
an intermediate shaft (140) configured to transfer the torque of the motor (130) to the slip ring (110) and the optical rotary joint (120), and disposed with the slip ring (110) and the optical rotary joint (120) in parallel to synchronize rotations of the motor (130), the slip ring (110), and the optical rotary joint (120),
wherein the intermediate shaft (140) includes a main drive part (141) disposed on the intermediate shaft (140) to receive the torque from the motor pulley (131) of the motor (130), and component drive parts (142) disposed on the intermediate shaft (140) on which the main drive part (141) is disposed to transfer the torque transferred from the main drive part (141) to the slip ring (110) and the optical rotary joint (120) respectively, and
timing belts connect the motor pulley (131) and a pulley of the main drive part (141) to each other, and connect pulleys of the component drive parts (142) and pulleys of the slip ring (110) and the optical rotary joint (120) to each other to transfer the torque.

12. The apparatus of claim 11, wherein the intermediate shaft (140) is configured to allow expansion of the integrated scanning system by adding a new component drive part (142) to transfer the torque to an additional component.

13. The apparatus of claim 12, wherein any one of the motor pulley (131) of the motor (130) and the pulley of the main drive part (141) further includes an unidirectional bearing (143) that transfers power during acceleration of the motor (130), but serves as a bearing during deceleration of the motor (130) so as not to transfer power, thereby improving the uniformity of the motor speed.

14. A method of improving rotation stability and expandability of an integrated scanning system including a slip ring (110), an optical rotary joint (120), a motor (130), and an intermediate shaft (140), the method comprising:
(1) a step in which a pulley of a main drive part (141) of the intermediate shaft (140) receives torque through a motor pulley (131) of the motor (130);
(2) a step in which a plurality of component drive parts (142) disposed and fixed on the intermediate shaft (140) rotates on the intermediate shaft (140) by the torque of the main drive part (141); and
(3) a step in which the plurality of component drive parts (142) disposed and fixed on the intermediate shaft (140) transfers the torque to the slip ring (110) and the optical rotary joint (120) respectively.

15. The method of claim 14, wherein the intermediate shaft (140) is disposed with the slip ring (110) and the optical rotary joint (120) in parallel.

16. The method of claim 15, wherein the intermediate shaft (140) synchronizes rotations of the motor (130), the slip ring (110), and the optical rotary joint (120).

17. The method of any one of claim 14, wherein slip ring (110) and the optical rotary joint (120) are configured to be disposed independently on the same shaft to receive torque from the respective component drive parts (142) disposed on the intermediate shaft (140).

18. The method of claim 17, wherein the intermediate shaft (140) is configured to allow expansion of the integrated scanning system by adding a new component drive part (142) to transfer the torque to an additional component.

19. The method of claim 17, wherein the intermediate shaft (140) serves to improve uniformity of a motor speed by adjusting a gear ratio between the motor pulley (131) of the motor (130) and a pulley of the main drive part (141).

20. The method of claim 19, wherein any one of the motor pulley (131) of the motor (130) and the pulley of the main drive part (141) further includes an unidirectional bearing (143) that transfers power during acceleration of the motor (130), but serves as a bearing during deceleration of the motor (130) so as not to transfer power, thereby improving the uniformity of the motor speed.
